(19) United States
(12) Patent Application Publication
(10) Pub. No.: US 2021/0364489 A1
(43) Pub. Date: Nov. 25, 2021

(54) PORTABLE SENSOR DEVICE FOR RAPID DETECTION OF HEAVYMETAL IONS AND METHODS THEREFOR

(71) Applicant: Board of Trustees of the Leland Stanford Junior University, Stanford, CA (US)

(72) Inventors: Yi Cui, Stanford, CA (US); Hiang Kwee Lee, Stanford, CA (US); Wenxiao Huang, Stanford, CA (US); Yusheng Ye, Stanford, CA (US)

(73) Assignee: Board of Trustees of the Leland Stanford Junior University, Stanford, CA (US)

(21) Appl. No.: 17/243,314

(22) Filed: Apr. 28, 2021

Related U.S. Application Data
(60) Provisional application No. 63/027,192, filed on May 19, 2020.

(51) Int. Cl. G01N 33/18; G01N 21/78

(52) U.S. Cl. CPC G01N 33/1813; B01L 2300/166; G01N 21/78

(57) ABSTRACT

Example implementations include a sensor device with a superhydrophobic sensor panel having an abraded first planar surface and a second planar surface opposite to the first planar surface, and a metallic heating element adjacent to the second planar surface of the superhydrophobic sensor panel. Example implementations also include a method of detecting a concentration of heavy metal ions in a solution, by separating a target solute of a target microdroplet from the target micropdroplet, identifying a distribution area of at least one heavy metal ion in an image of the target solute, generating a heavy metal ion concentration quantity based on the distribution area, generating a composite image including an indication of the distribution area, and presenting an indication of at least one of the heavy metal ion concentration quantity and the composite heavy metal ion image. Example implementations also include a method of manufacturing a heavy metal ion sensor device, by abrading a first planar surface of a superhydrophobic sensor panel, depositing a metallic layer on a nonconductive substrate, and contacting the metallic layer to a second planar surface of the sensor panel opposite to the first planar surface.

### CROSS-REFERENCE TO RELATED PATENT APPLICATIONS

This application claims priority to U.S. Provisional Patent Application Ser. No. 63/027,192, entitled “ULTRATRACE AND COLLECTIVE VISUAL DETECTION OF HEAVY METAL IONS BY THEIR SULFIDATION ON SUPERHYDROPHOBIC CONCENTRATOR (SPOT),” filed May 19, 2020, the contents of such application being hereby incorporated by reference in its entirety and for all purposes as if completely and fully set forth herein.

### TECHNICAL FIELD

The present implementations relate generally to sensor devices, and more particularly to a portable sensor device for rapid detection of heavy metal ions and methods therefor.

### BACKGROUND

One in three people worldwide do not have access to safe drinking water. Notably, heavy metal ions (HMIs) are major water pollutants threatening human health due to their severe toxicity, even at trace levels. Efficient HMI detection thus plays a major defense against metal poisoning by enabling early pollution warning and efficient regulatory enforcement. However, it remains a formidable challenge to accurately detect these pollutants on-site at ultratrace level in a cost and time-effective manner.

Heavy metal ions are toxic environmental pollutants that readily react with biological matter to disrupt their molecular structures and functions, causing serious toxicological and carcinogenic effects to numerous body systems and vital organs. These health threats are further aggravated by the continuous accumulation of heavy metals in the ecosystem due to their non-biodegradability, as well as increasing anthropogenic pollutions caused by rapid global industrialization. Ultrasensitive detection of heavy metal ions thus serves as a major line of defense against heavy metal poisoning by enabling efficient regulatory enforcement and early warning of potential pollutions. However, conventional systems are impeded by the need for sophisticated and expensive instrumentation, trained operators, and tedious pre-treatment processes. Consequently, these methods are inapt for routine and frequent testing and lack portability for on-site detection.

The risk of heavy metal poisoning is clear. However, pollution problems are often uncovered only when health issues develop. Thus, it is absolutely critical to carry out easy-to-operate routine and frequent tests for the presence of heavy metal ions, especially in potable water. This would be particularly important for underserved domestic communities, as well as Global South countries facing heavy metal pollution problems arising from rapid industrialization and urbanization. Thus, there is a clear need to make heavy metal ion testing decentralized, time-efficient, and cost-effective while maintaining high detection accuracy and sensitivity.

### SUMMARY

A heavy metal ion sulfidation on superhydrophobic concentrator sensor device in accordance with present implementations can integrates a sulfidation chemical process to react with heavy metal ions, and a concentrator structure to collect heavy metal ions for increased visual detection. The sulfidation process can chemically recognize heavy metal ions with high reactivity due to low solubility constant of heavy metal sulfides, and can be generically selective towards five target ionic species. The generated insoluble, dark heavy metal sulfide can thus be the basis for visual detection. Further, in some implementations, an integrated analyte concentrator using superhydrophobic platform can, upon droplet drying, continuously accumulate heavy metal sulfide from the entire droplet volume into a small area. The increase in particle surface density in turn creates large, visible particle clusters for sensitive read-out by the naked eyes and/or optical devices, even at highly diluted concentrations. It is to be understood that that optical devices can detect concentration orders of magnitude lower than unmagnified or naked eye visual detection.

In some implementations, a sensor device in accordance with present implementations can detect individual toxic species down to a detection limit of 0.1 nanomolar and can demonstrate a wide quantification range over several order of magnitudes, based on a sample microdroplet of approximately 5 μL.

In some implementations, a detection limit of 0.1 nanomolar is 3- to 6-orders of magnitudes more sensitive than respective permissible limits recommended by the World Health Organization (WHO) and the US Environmental Protection Agency (USEPA). Moreover, detection in accordance with present implementations also enables the concurrent quantification of multiple heavy metal ion species into a group of “dark heavy metal ions,” the presence of which can indicate general water safety. a system in accordance with present implementations can include one or more of a heavy metal ion sensor device, a miniature droplet heater, a portable microscope and a custom mobile application for direct concentration readout. In some implementations, the system can be highly portable and efficient to detect heavy metal ions in real/artificial sample matrices (soil, tap water and industrial wastewater), at high speeds of less than 8 min., and high accuracy of approximately 90%.

Example implementations include a sensor device with a superhydrophobic sensor panel having an abraded first planar surface and a second planar surface opposite to the first planar surface, and a metallic heating element adjacent to the second planar surface of the superhydrophobic sensor panel.

Example implementations also include a sensor device with a nonconductive substrate adjacent to the metallic layer, where the metallic layer is adjacent to the superhydrophobic sensor layer at a first planar surface thereof, and adjacent to the nonconductive substrate at a second planar surface thereof.

Example implementations also include a sensor device where the abraded first planar surface includes surface features each having contact angles of 150 degrees or greater.

Example implementations also include a sensor device where the superhydrophobic sensor panel includes poly(tetrafluoroethylene) (PTFE).

Example implementations also include a sensor device where the metallic heating element includes nickel.

Example implementations also include a sensor device with a first electrical lead in contact with the metallic heating element at a first end of the metallic heating element, and a second electrical lead in contact with the metallic heating element at a second end of the metallic heating element distal to the first end of the metallic heating element.

Example implementations also include a sensor device with a battery device operatively coupled to the first electrical lead and the second electrical lead, and configured to cause heating of the metallic heating element by applying an electrical current thereto.

Example implementations also include a method of detecting a concentration of heavy metal ions in a solution, by separating a target solute of a target microdroplet from the target micropdroplet, identifying a distribution area of at least one heavy metal ion in an image of the target solute, generating a heavy metal ion concentration quantity based on the distribution area, generating a composite image including an indication of the distribution area, and presenting an indication of at least one of the heavy metal ion concentration quantity and the composite heavy metal ion image.

Example implementations also include a method of further depositing the target micropdroplet on an abraded first planar surface of a superhydrophobic sensor panel.

Example implementations also include a method where the separating the target solute from the target micropdroplet by separating the target solute of the target microdroplet from the target micropdroplet by heating the target microdroplet to a temperature between 35 degrees C. and 105 degrees C.

Example implementations also include a method the at least one heavy metal ion is at least one of lead, nickel, chromium, cobalt, and copper.

Example implementations also include a method of further adding a reaction solution including at least one of sodium sulfide and sodium carbonate to the target microdroplet.

Example implementations also include a method of further capturing the image of the target solute at microscopic magnification by a microscopic imaging device.

Example implementations also include a method where the identifying the distribution area of the heavy metal ion in the image of the target solute includes identifying the distribution area of the heavy metal ion in the image of the target solute by identifying a number of pixels associates with the distribution area of the heavy metal ion in the image.

Example implementations also include a method where the generating the heavy metal ion concentration quantity based on the distribution area includes generating the heavy metal ion concentration quantity based on the distribution area by a nonlinear correlation between distribution area and heavy metal ion concentration.

Example implementations also include a method of manufacturing a heavy metal ion sensor device, by abrading a first planar surface of a superhydrophobic sensor panel, depositing a metallic layer on a nonconductive substrate, and contacting the metallic layer to a second planar surface of the sensor panel opposite to the first planar surface.

Example implementations also include a method where the abrading the first planar surface of the superhydrophobic sensor panel includes abrading the first planar surface of the superhydrophobic sensor panel at contact angles of 150 degrees or greater.

Example implementations also include a method where the abrading the first planar surface of the superhydrophobic sensor panel includes abrading the first planar surface of the superhydrophobic sensor panel by friction contact at the first planar surface with an abrasive solid.

Example implementations also include a method where the superhydrophobic sensor panel includes poly(tetrafluoroethylene) (PTFE).

Example implementations also include a method where the metallic layer includes nickel.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1A illustrates an example cross-sectional view of an example portable sensor device for rapid detection of heavy metal ions, in accordance with present implementations.

FIG. 1B illustrates an example plan view of a portable sensor device for rapid detection of heavy metal ions further to the example cross-sectional view of FIG. 1A.

FIG. 2A illustrates a first example distribution area of heavy metal ions on a portable sensor device for rapid detection of heavy metal ions, in accordance with present implementations.

FIG. 2B illustrates a second example distribution area of heavy metal ions on a portable sensor device for rapid detection of heavy metal ions, in accordance with present implementations.

FIG. 2C illustrates a third example distribution area of heavy metal ions on a portable sensor device for rapid detection of heavy metal ions, in accordance with present implementations.

FIG. 3 illustrates an example system including a portable sensor device for rapid detection of heavy metal ions, in accordance with present implementations.

FIG. 4 illustrates an example system memory in accordance with a portable sensor device for rapid detection of heavy metal ions, in accordance with present implementations.

FIG. 5 illustrates an example presentation of a detection of heavy metal ions in a distribution areas of a portable sensor device for rapid detection of heavy metal ions, in accordance with present implementations.

FIG. 6 illustrates an example method of depositing a target analyte at a portable sensor device for rapid detection of heavy metal ions, in accordance with present implementations.

FIG. 7 illustrates an example method of preprocessing an image of a target analyte at a portable sensor device for rapid detection of heavy metal ions, further to the example method of FIG. 6.

FIG. 8 illustrates an example method of sensing and presenting a concentration of a target analyte at a portable sensor device for rapid detection of heavy metal ions further to the example method of FIG. 7.

FIG. 9 illustrates an example method of manufacturing a portable sensor device for rapid detection of heavy metal ions, in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Sensor devices and system in accordance with present implementations provide a convenient and cost-effective approach to detect heavy metal ions directly at the point of concern. Compared to traditional analytical techniques, on-site detection provides timely information on water safety necessary for immediate decision/counteractions to be taken, an advantage especially important in remote areas with no access to conventional testing facilities.

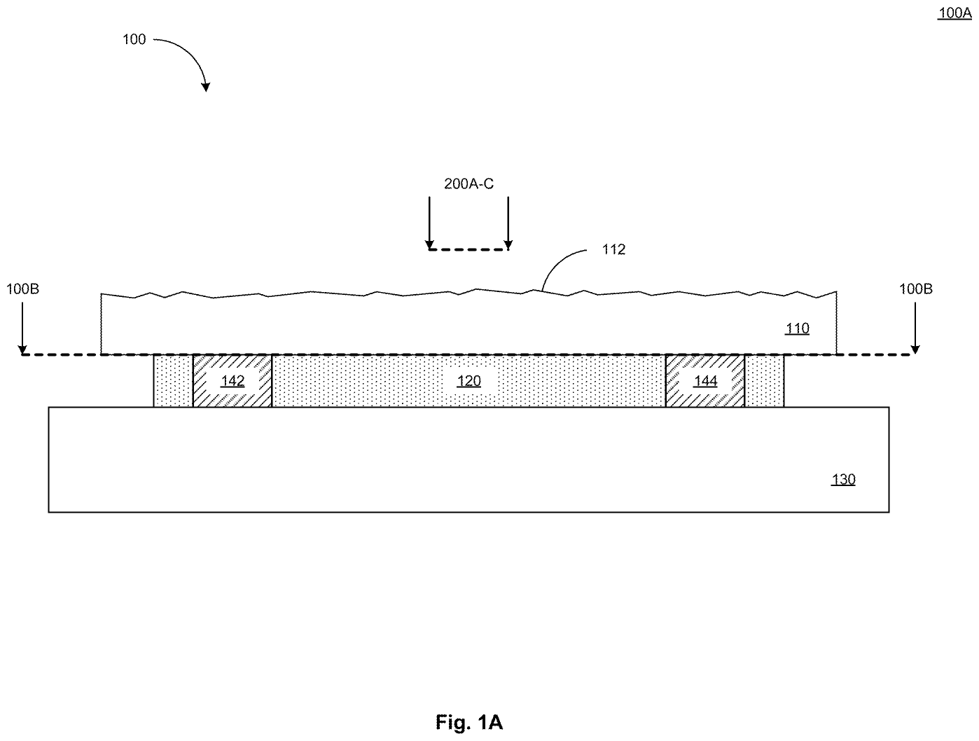

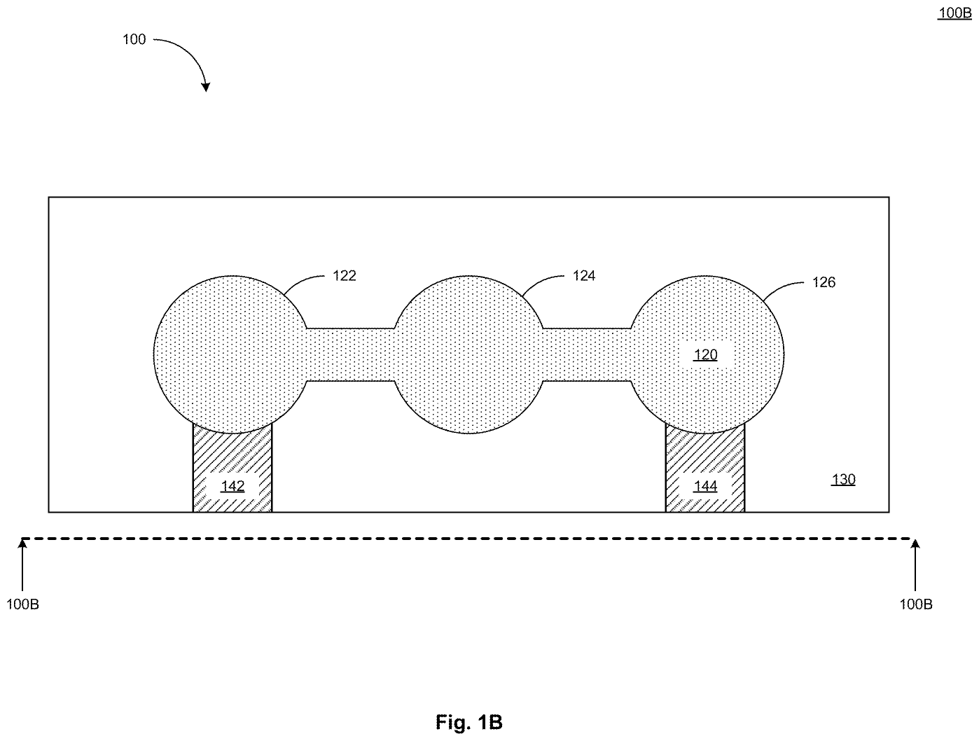

FIG. 1A illustrates an example cross-sectional view of an example portable sensor device for rapid detection of heavy metal ions, in accordance with present implementations. As illustrated by way of example in FIG. 1A, an example portable sensor device for rapid detection of heavy metal ions 100 in cross-sectional view 100A includes a superhydrophobic sensor panel 110, a metallic heating element 120, a nonconductive substrate 130, and conductive leads 142 and 144.

The superhydrophobic sensor panel 110 is operable to concentrate solute including heavy metal ions. In some implementations, the superhydrophobic sensor panel 110 is or includes a hydrophobic material, superhydrophobic material, ultrahydrophobic material, or the like. In some implementations, the superhydrophobic sensor panel 110 is or includes poly(tetrafluoroethylene) (PTFE). In some implementations, the superhydrophobic sensor panel is a PTFE sheet, film, or the like. It is to be understood that PTFE is hydrophobic, chemically-inert, and provides a white background to facilitate identification of dark metal sulfides. It is to be further understood that hydrophobic characteristics of PTFE can be at least partially based on contact angle of the PTFE at a surface thereof. As one example, a contact angle of PTFE can be approximately 102 degrees in an unrefined state. In some implementations, the PTFE is refined by abrasion or the like to advantageously generate the sensor surface 112 with superhydrophobic, ultrahydrophobic, or like characteristics. In some implementations, the sensor surface 112 of the superhydrophobic sensor panel 110 includes an abraded, uneven, or like surface including a plurality of surface variations. The surface variations can be associated with particular contact angles between a microdroplet or the like containing solute and the sensor surface 112. In some implementations, contact angles of the sensor surface are between 151 degrees and 153 degrees. As one example, a mean or median contact angle of the sensor surface 112 can be 152 degrees. As one example, contact angles of the sensor surface 112 can be 150 degrees or greater.

The metallic heating element 120 is operable to heat one or more of the superhydrophobic sensor panel 110 and the sensor surface 112 to evaporate any liquid present on the sensor surface 112. In some implementations, the metallic heating element 120 is or includes nickel. In some implementations, the metallic heating element 120 is in direct contact with the sensor panel 120 at a planar surface thereof. In some implementations, the planar surface of the sensor panel 110 in contact with the metallic heating element 120 is opposite to the sensor surface 112. As one example, the metallic heating element can be approximately 100 nm thick to provide a thin heating component to advantageously support a smaller and more portable device. As one example, the metallic heating element can have a length of approximately 19 mm.

The nonconductive substrate 130 is disposed in contact with the metallic heating element 120. In some implementations, the metallic heating element 120 is or includes a metal deposited on the nonconductive substrate further to formation by evaporation, plating, deposition, lithography, sputtering, or the like. As one example, the nonconductive substrate can be glass or the like. It is to be understood that the metallic heating element 120 can be formed directly on the sensor panel 110, and need not be formed on the nonconductive substrate 130. It is to be further understood that where the metallic heating element 120 is formed on the sensor panel 110, the nonconductive substrate 130 is optional or can be omitted from the portable sensor device for rapid detection of heavy metal ions 100.

The conductive leads 142 and 144 are disposed in contact with the metallic heating element 120, and are operable to provide electrical energy to the metallic heating element 120. In some implementations, the conductive leads 142 and 144 are or include copper. In some implementations, the conductive lead 142 is located at a first end of the metallic heating element 120, and the conductive lead 144 is located at an opposite end, distal end, opposite portion, distal portion, or the like of the metallic heating element 120.

FIG. 1B illustrates an example plan view of a portable sensor device for rapid detection of heavy metal ions further to the example cross-sectional view of FIG. 1A. As illustrated by way of example in FIG. 1B, an example portable sensor device for rapid detection of heavy metal ions 100 in plan view 100B includes the metallic heating element 120, the nonconductive substrate 130, and the conductive leads 142 and 144.

In some implementations, the metallic heating element 120 includes heat concentrator regions 122, 124 and 126. In some implementations, the heat concentrator regions 122, 124 and 126 are each substantially circular portions of the metallic heating element 120 and are disposed linearly along an extending direction of the metallic heating element 120. It is to be understood that the heat concentrator regions 122, 124 and 126 can be or include nickel and can be integrably formed with the metallic heating element 120. It is to be further understood that the heat concentrator regions 122, 124 and 126 can advantageously allow the metallic heating element 120 to apply a greater amount of heat to the sensor panel 110 and effect a more rapid drying process for any liquid, micropdroplet, or the like present on the sensor surface 112 of the sensor panel 110. In some implementations, a diameter of each of the heat concentrator regions 122, 124 and 126 can be approximately 3 mm. It is to be understood that the heat concentrator regions 122, 124 and 126 can be other elliptical or polygonal shapes other than substantially circular, and can have a diameter, width, or the like, greater or less than 3 mm to accommodate a planar surface area of the sensor panel 110, where the planar surface area of the sensor panel 110 deviates from a rectangular shape. In some implementations, each of the heat concentrator regions 122, 124 and 126 can be separated by a distance of approximately 5 mm. In some implementations, the metallic heating element 120 has a width of 1 mm.

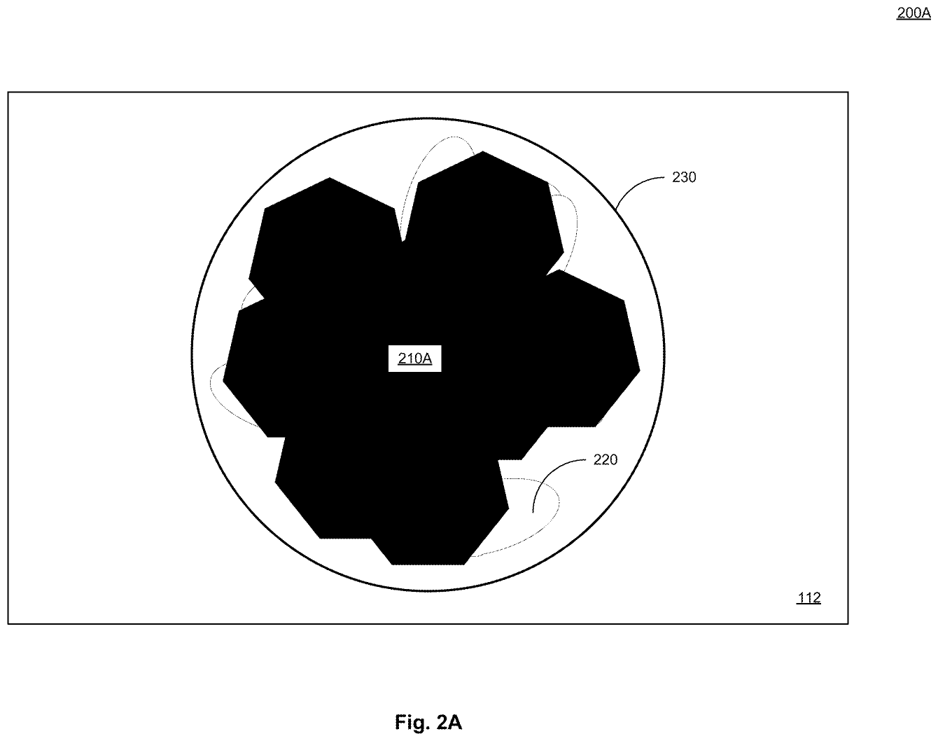

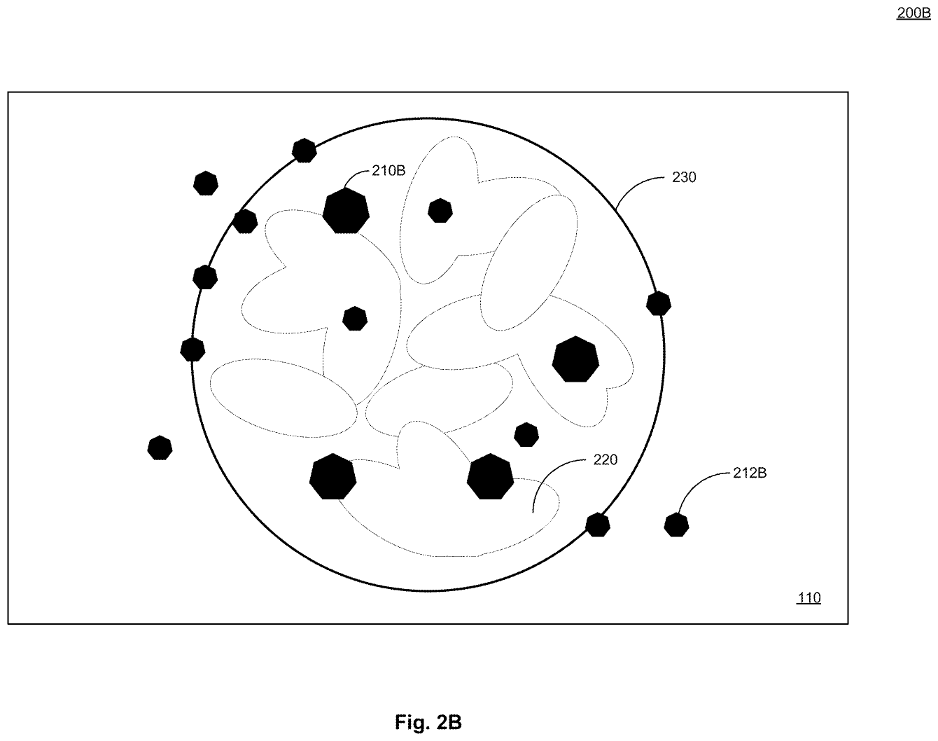

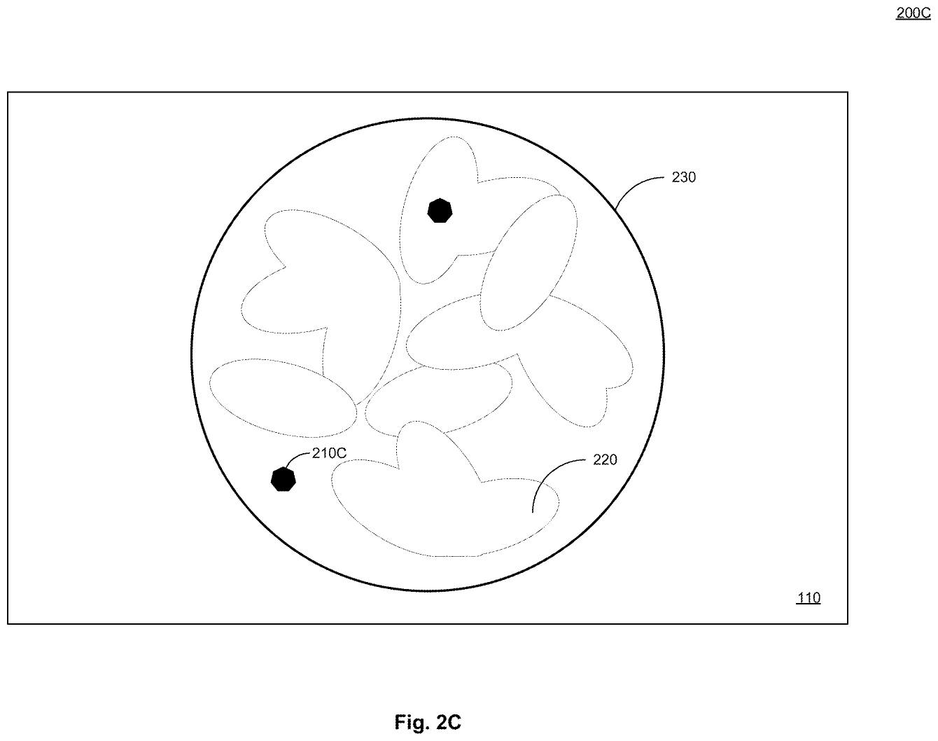

FIGS. 2A-C illustrate example distributions of heavy metal ions on a portable sensor device for rapid detection of heavy metal ions, in accordance with present implementations. As illustrated by way of example in FIGS. 2A-C, example distributions of heavy metal ions on a portable sensor device for rapid detection of heavy metal ions 200A-C each include sodium salts 220 and a spot ring 230 disposed on the sensor surface 112.

The sodium salts 220 are white, colorless salts that remain on the sensor surface 112 after evaporation of a liquid, microdroplet, or the like containing at least one of sodium sulfide and sodium carbonate. It is to be understood that the white and colorless visual appearance of the sodium slats 220 advantageously improves visual detection of dark-colored heavy metal ions due to the contrasting color, luminosity, and like properties of the target heavy metal ions. As one example, visually detectable heavy metal ions include Pb′, Ni′, Cr′, Cu′ and Co′. It is to be understood that high reactivity of heavy metal 113 sulfidation can be driven by the low Ksp of corresponding metal sulfides, whereby S²⁻ ions spontaneously arrest these cations to generate insoluble heavy metal sulfide. As one example, Ksp of PbS is 10⁻²⁷ M, Ksp of NiS is 10⁻¹⁹M, Ksp of CuS is 10⁻³⁶ M, and Ksp of CoS is 10⁻²¹ M.

The spot ring 230 indicates an evaporation region associated with an evaporated microdroplet or the like including the sodium salts 220 and optionally including heavy metal ions. It is to be understood that heating hygroscopic Na₂S can leave a liquid ring around the spot. It is to be further understood that that spot ring 230 has a negligible effect on SPOT-based quantification of heavy metal ions.

FIG. 2A illustrates a first example distribution area of heavy metal ions on a portable sensor device for rapid detection of heavy metal ions, in accordance with present implementations. As illustrated by way of example in FIG. 2A, a first example distribution area 200A includes a heavy metal ion deposition area 210A, the sodium salts 220 and the spot ring 230. As one example, the heavy metal ion deposition area 210A is associated with a maximum heavy metal ion concentration level of 10⁻³ M. In some implementations, at the maximum concentration level, a distribution area is substantially a single contiguous area of deposited heavy metal ions on the sensor surface 112.

FIG. 2B illustrates a second example distribution area of heavy metal ions on a portable sensor device for rapid detection of heavy metal ions, in accordance with present implementations. As illustrated by way of example in FIG. 2B, a second example distribution area 200B includes heavy metal ion deposition portions 210B, peripheral heavy metal ion deposition portions 212B, the sodium salts 220 and the spot ring 230. As one example, the heavy metal ion deposition portions 210B and 212B are associated with a higher heavy metal ion concentration level of 10⁻⁷ M. In some implementations, the spot ring 230 is a boundary indicating an outer periphery for measurement of a distribution area. Thus, in some implementations, the peripheral heavy metal ion deposition portions 212B are not incorporated into a measure of distribution area of heavy metal ions, and only the heavy metal ion deposition portions 210B within the spot ring 230 are incorporated into the measure of distribution area of heavy metal ions.

FIG. 2C illustrates a third example distribution area of heavy metal ions on a portable sensor device for rapid detection of heavy metal ions, in accordance with present implementations. As illustrated by way of example in FIG. 2C, a third example distribution area 200C includes a heavy metal ion deposition area 210C, the sodium salts 220 and the spot ring 230. As one example, the heavy metal ion deposition area 210C is associated with a minimum heavy metal ion concentration level of 10⁻¹⁰ M. Thus, in some implementations, a portable sensor device in accordance with present implementations can advantageously visually quantify the presence of up to five types of common heavy metal ions Pb²⁺, Ni²⁺, Cr²⁺, Cu²⁺ and Co²⁺ to a concentration of 10⁻¹⁰ M. It is to be understood that that the minimum concentration of 10⁻¹⁰ M is multiple orders of magnitude less than a minimum safe concentration level for one or more of the heavy metal ions with respect to one or more safety regulations. For example, USEPA limits on common heavy metal ions such as lead (Pb), nickel (Ni), chromium (Cr), cobalt (Co) and copper (Cu) can range from 10⁻⁸ to 10⁻⁵ M. It is to be understood that the portable sensor device in accordance with present implementations can advantageously visually quantify the presence of these heavy metal ions without complex and expensive instrumentation, and without complex processes like, for example, atomic absorption spectroscopy (AAS) and inductively coupled plasma-mass spectrometry (ICP-MS). This advantage is especially important in remote areas with no access to testing facilities.

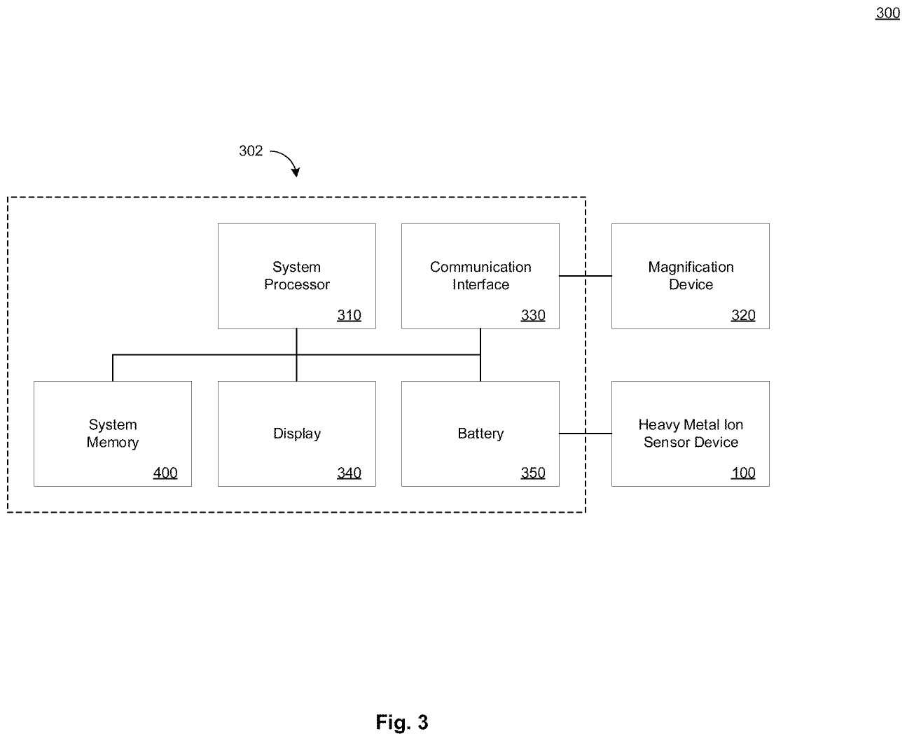

FIG. 3 illustrates an example system including a portable sensor device for rapid detection of heavy metal ions, in accordance with present implementations. As illustrated by way of example in FIG. 3, an example system 300 includes the heavy metal ion sensor device 100, a system processor 310, a magnification device 320, a communication interface 330, a display 340, a battery device 350, and a system memory 400.

In some implementations, one or more of the system processor 310, the magnification device 320, the communication interface 330, the display 340, the battery device 350, and the system memory 400 is associated with, integrated with, coupled to, or the like, an electronic device 302. In some implementations, the electronic device 302 is a mobile electronic device. In some implementations, the electronic device 302 includes but is not limited to a smartphone, mobile device, wearable mobile device, tablet computer, desktop computer, laptop computer, cloud server, local server, and the like.

The system processor 310 is operable to execute one or more instructions associated with input from one or more of the magnification device 320 and the communication interface 330. In some implementations, the system processor 310 is an electronic processor, an integrated circuit, or the like including one or more of digital logic, analog logic, digital sensors, analog sensors, communication buses, volatile memory, nonvolatile memory, and the like. In some implementations, the system processor 310 includes but is not limited to, at least one microcontroller unit (MCU), microprocessor unit (MPU), central processing unit (CPU), graphics processing unit (GPU), physics processing unit (PPU), embedded controller (EC), or the like. In some implementations, the system processor 310 includes a memory operable to store or storing one or more instructions for operating components of the system processor 310 and operating components operably coupled to the system processor 310. In some implementations, the one or more instructions include at least one of firmware, software, hardware, operating systems, embedded operating systems, and the like. It is to be understood that the system processor 310 or the device 310 generally can include at least one communication bus controller to effect communication between the system processor 310 and the other elements of the system 300 or the device 302.

The magnification device 320 is operatively coupled to the communication interface 330 and is operable to capture at least one visual image of the heavy metal ion sensor device 100. In some implementations, the magnification device 320 is operable to capture one or more images in accordance with the example distributions of heavy metal ions 2A-C. It is to be understood that the system 300 can optionally include the magnification device 320 where the system 300 or the electronic device includes, is coupled to, or the like, a camera device capable of magnification. In some implementations, the magnification device 320 is a microscope, handheld microscope, portable microscope, or the like. In some implementations, the magnification device 320 includes an output, interface, or the like compatible with the communication interface 330.

The communication interface 330 is operable to communicatively couple the system processor 310 to at least one device external to the system processor 310. In some implementations, the communication interface 330 is operable to communicate one or more instructions, signals, conditions, states, or the like between one or more of the system processor 310 and the external device. In some implementations, the communication interface 330 includes one or more digital, analog, or like communication channels, lines, traces, or the like. As one example, the communication interface 330 is or includes at least one serial or parallel communication line among multiple communication lines of a communication interface. In some implementations, the communication interface 330 is or includes one or more wireless communication devices, systems, protocols, interfaces, or the like. In some implementations, the communication interface 330 includes one or more logical or electronic devices including but not limited to integrated circuits, logic gates, flip flops, gate arrays, programmable gate arrays, and the like. In some implementations, the communication interface 330 includes ones or more telecommunication devices including but not limited to antennas, transceivers, packetizers, wired interface ports, and the like. It is to be understood that any electrical, electronic, or like devices, or components associated with the communication interface 330 can also be associated with, integrated with, integrable with, replaced by, supplemented by, complemented by, or the like, the system processor 310 or any component thereof.

The display 340 is operable to display one or more chemicals, chemical concentrations, chemical characteristics, and the like, associated with one or more heavy metal ions detected by the system 300 or the electronic device 302. In some implementations, the display device 340 includes an electronic display. In some implementations, the electronic display includes a liquid crystal display (LCD), a light-emitting diode (LED) display, an organic light-emitting diode (OLED) display, or the like. In some implementations, the display device 340 is housed at least partially within the electronic device 302.

The battery 350 includes one or more electrical, electronic, electromechanical, electrochemical, or like devices or systems for at least one of receiving, storing and distributing input power. In some implementations, the battery 350 includes one or more stacks of batteries. In some implementations, the battery 350 includes lithium-ion or like energy storage. In some implementations, the battery 350 is integrated with, integrable with, or separable from the example system 300. In some implementations, the battery 350 includes a plurality of battery units variously or entirely integrated with, integrable with, or separable from the system 300. In some implementations, the system 300 is operatively coupled to an external power source separate from the battery 350. In some implementations, the battery 350 is operatively coupled to the heavy metal ion sensor device 100 by the conductive leads 142 and 144. In some implementations, the battery 350 supplies a low-amperage electrical current to the heavy metal ion sensor device 100 to advantageously provide evaporative heat to the liquid, microdroplet, or the like present thereon, while preventing overheating that may obscure visual identification of various heavy metal ions present therein.

The system memory 400 is operable to store data associated with the system 300. In some implementations, the system memory 400 includes ones or more hardware memory devices for storing binary data, digital data, or the like. In some implementations, the system memory 400 includes one or more electrical components, electronic components, programmable electronic components, reprogrammable electronic components, integrated circuits, semiconductor devices, flip flops, arithmetic units, or the like. In some implementations, the system memory 400 includes at least one of a non-volatile memory device, a solid-state memory device, a flash memory device, and a NAND memory device. In some implementations, the system memory 400 includes one or more addressable memory regions disposed on one or more physical memory arrays. In some implementations, a physical memory array includes a NAND gate array disposed on a particular semiconductor device, integrated circuit device, printed circuit board device, and the like.

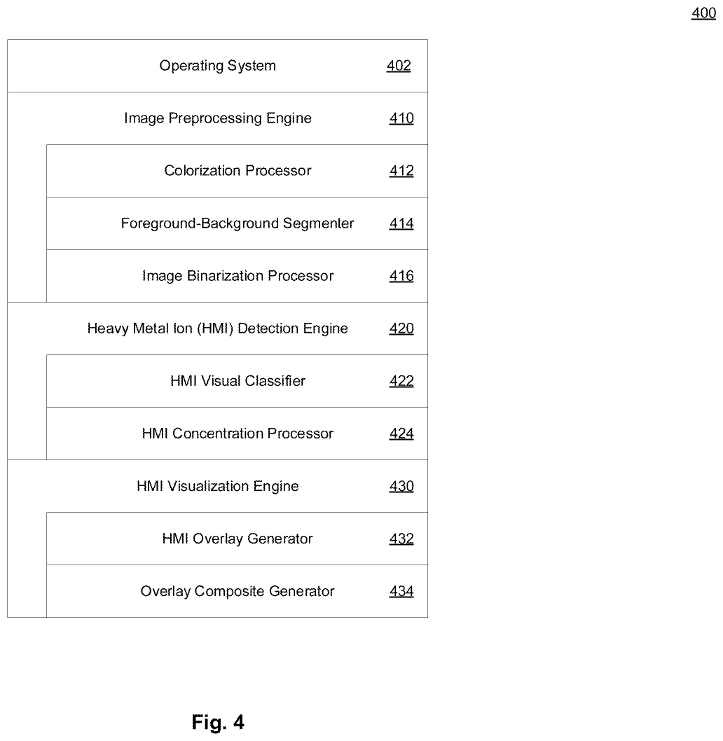

FIG. 4 illustrates an example system memory in accordance with a portable sensor device for rapid detection of heavy metal ions, in accordance with present implementations. As illustrated by way of example in FIG. 4, an example system memory 400 includes an operating system 402, an image preprocessing engine 410, a heavy metal ion detection engine 420, and a heavy metal ion visualization engine 430.

The operating system 402 includes one or more instructions for operating one or more components associated with at least with the system 300, for generating at least presentation 500, and for performing one or more steps associated at least with methods 600, 700 and 800 in accordance with present implementations. In some implementations, the operating system 402 is at least one of an embedded operating system, a mobile operating system, a desktop operating system, a server operating system, or the like. It is to be understood that the operating system 402 includes, is integrable with, is couplable with, is integrated with, is coupled to, or the like one or more interfaces, drivers, executables, instructions, or the like, for operating one or more of the components of the system 300. In some implementations, the operating system 402 is operable to capture a visual image from a camera device associated therewith. In some implementations, the operating system 402 is operable to capture a magnified visual image from at least one of a camera device and a magnification device 320 associated therewith.

The image preprocessing engine 410 includes one or more instructions for receiving a visual image and generating a preprocessed visual image supporting detection of heavy metal ions. In some implementations, the image preprocessing engine 410 includes one or more of a colorization processor 412, a foreground-background segmenter 414, and an image binarization processor 416.

The colorization processor 412 includes one or more instructions for modifying one or more color characteristics of a received visual image. In some implementations, the colorization processor 412 converts a color image to a grayscale image by converting pixels of the received image from a color pixel to a grayscale pixel. As one example, a color pixel can include RGB or CMYK color components. As another example, a grayscale pixel can include a single luminosity component. As another example, a grayscale pixel can have an 8-bit luminosity component from 0 to 255, or a higher luminosity component of 16 or 24 bits, for example. A luminosity component having an 8-bit or higher bit size can advantageously support visual detection of heavy metal ions at concentration levels beyond the capacity of detection by human visual perception alone, beyond the capacity of detection without preprocessing, or beyond the capacity of detection without magnification, or any combination thereof.

The foreground-background segmenter 414 includes one or more instructions for separating a sensor panel region from a maximum potential distributin area associated with heavy metal ions. In some implementations, the foreground-background segmenter 414 spatially segments the received visual image based on a distance from a center point of an image or other focal point of the received visual image. As one example, the foreground-background segmenter 414 can segment a background portion of an image greater than a particular threshold radius from a center point, focal point, or the like, of the received visual image. In some implementations, the foreground-background segmenter 414 segments the foreground of the received image from the background of the received image by one or more features of the received image. As one example, the foreground-background segmenter 414 can segment the foreground of the received image from the background of the received image by the spot ring 230. As another example, the foreground-background segmenter 414 can classify at least a portion of the received visual image within the spot ring 230 as a foreground, and at least a portion of the received visual image outside the spot ring as background. In some implementations, the foreground-background segmenter 414 inverts pixels to advantageously increase the detectability of heavy metal ion pixels in the received visual image. As one example, the foreground-background segmenter 414 can convert darker pixels to lighter pixels, and can correspondingly convert lighter pixels to darker pixel by a luminosity inversion process.

The image binarization processor 416 includes one or more instructions for converting a pixel to a one-bit pixel. In some implementations, a one-bit pixel has two possible values, either white or black. In some implementations, the white or black pixels respectively correspond to “1” and “0” pixel values. In some implementations, the image binarization processor 416 includes one or more instructions to define and execute a binarization threshold value for binarization of a received visual image. As one example, a binarization value can be a particular luminosity value. As another example, a particular luminosity value can be 155. It is to be understood that the luminosity value can advantageously visually isolate pixels associated with heavy metal ions from pixels associated with sodium salts and a sensor surface.

The heavy metal ion detection engine 420 includes one or more instructions for detecting position and concentration of heavy metal ions present in a preprocessed visual image. In some implementations, the heavy metal ion detection engine 420 includes one or more of a heavy metal ion visual classifier 422 and a heavy metal ion concentration processor 424. The heavy metal ion visual classifier 422 includes one or more instructions for associating pixels values with either heavy metal ions or with objects and structures other than heavy metal ions. In some implementations, the heavy metal ion visual classifier 422 generates a classification object associating each pixel of the preprocessed visual image with a classification indicating that the pixel indicates presence of at least one heavy metal ion, or, a classification indicating that the pixel indicates absence of any heavy metal ions.

The heavy metal ion concentration processor 424 includes one or more instructions for generating a heavy metal concentration sensor output based on the classification object. In some implementations, the heavy metal ion concentration processor 424 generates, an aggregated value for, a sum of, or the like, all pixels indicating that the pixel indicates presence of at least one heavy metal ion. In some implementations, the heavy metal ion concentration processor 424 generates a concentration value for heavy metal ions present in the liquid, microdroplet, or the like, based on a correlation between the aggregated value, sum, or the like, and a function, or the like indicating a particular concentration. As one example, an correlation for the heavy metal concentration (H) and a distribution area (A) can be in accordance with Equation 1:

$$A = - 5.77 \; 879 {e}^{( \frac{- H}{9.94699} )} + 14.15412 \qquad Eqn . ( 1 )$$

The heavy metal ion visualization engine 430 includes one or more instructions for generating a visual indication of presence of heavy metal ions at a sensor surface. In some implementations, the heavy metal ion visualization engine 430 includes one or more of a heavy metal ion overlay generator 432 and an overlay composite generator 434.

The heavy metal ion overlay generator 432 includes one or more instructions for generating an overlay image indicating one or more positions of heavy metal ions at a sensor substrate. As one example, the heavy metal ion overlay generator 432 can generate a composite overlay based on a preprocessed visual image of a sensor surface. As another example, heavy metal ion overlay generator 432 can convert pixels of the preprocessed visual image indicating presence of at least one heavy metal ion to a first predetermined color, opacity, or combination thereof, and can convert pixels of the preprocessed visual image indicating absence of any heavy metal ion to a second predetermined color, opacity, or combination thereof. As one example, a first predetermined color and opacity combination can include a red color at a 100% opacity. As another example, a second predetermined color and opacity combination can include no color at a 0% opacity. The overlay composite generator 434 includes one or more instructions for combining the preprocessed visual image with the overlay image to generate a composite image. As one example, the overlay composite generator 434 can superimpose the overlay image on the preprocessed visual image such that locations of the preprocessed visual image associated with at least one type of heavy metal ion are displayed with a predetermined color, and all other portions of the preprocessed visual image are visible beneath the overlay image.

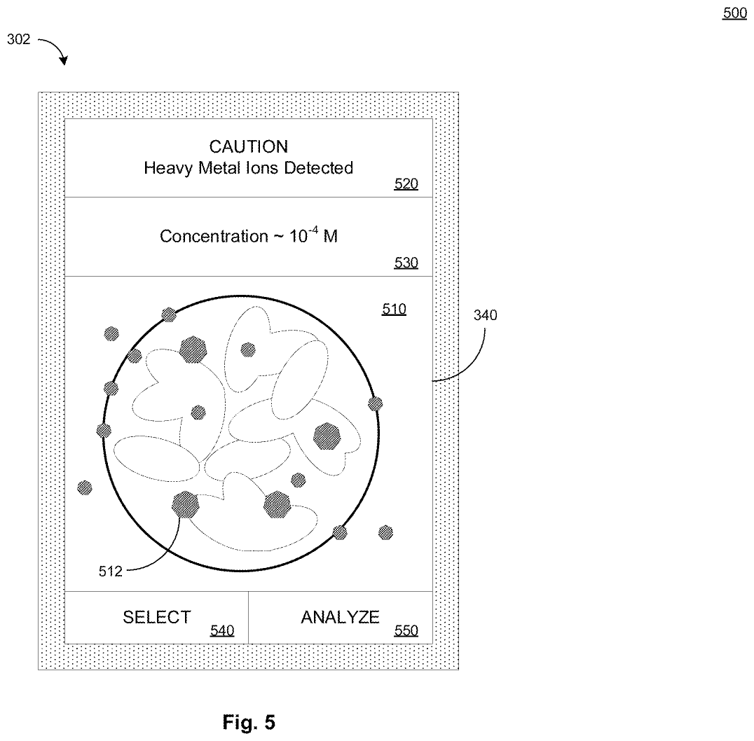

FIG. 5 illustrates an example presentation of a detection of heavy metal ions in a distribution areas of a portable sensor device for rapid detection of heavy metal ions, in accordance with present implementations. As illustrated by way of example in FIG. 5, an example presentation 500 at the display 340 of the electronic device 302 includes a heavy metal ion location indication display portion 510, a heavy metal ion presence indication display portion 520, a heavy metal ion concentration indication display portion 530, a heavy metal ion visual image selection interface affordance 540, and a heavy metal ion visual image processing interface affordance 540.

The heavy metal ion location indication display portion 510 is operable to present one or more of the received visual image, the preprocessed visual image, the overlay image, and the composite image. In some implementations, the heavy metal ion location indication display portion 510 includes one or more overlay object 512 having particular color and opacity characteristics in accordance with present implementations. In some implementations, the overlay objects 512 correspond to locations of heavy metal ions in the visual image.

The heavy metal ion presence indication display portion 520 is operable to indicate whether heavy metal ions are detected at the sensor device 100. In some implementations, the heavy metal ion presence indication display portion 520 indicates that heavy metal ions are detected in accordance with a technical threshold associated with a detectable concentration of heavy metal ions at or above 10⁻¹⁰ M. In some implementations, the heavy metal ion presence indication display portion 520 indicates that heavy metal ions are detected in accordance with a safety threshold associated with a safe concentration of heavy metal ions at or above 10⁻⁶ M, in accordance with one or more predetermined safety thresholds associated with one or more safety regulations.

The heavy metal ion concentration indication display portion 530 is operable to indicate a concentration associated with heavy metal ions are detected at the sensor device 100. In some implementations, the heavy metal ion concentration indication display portion 530 is operable to display an order of magnitude associated with concentrations between 10⁻³ M and 10⁻¹⁰ M by presenting the order of magnitude of the concentration as “˜ 10^{-N}” where N is the order of magnitude. In some implementations, the heavy metal ion concentration indication display portion 530 is operable to display an order of magnitude associated with concentrations either above 10⁻³ M or below 10⁻¹⁰ M by presenting the order of magnitude of concentration as either “>10⁻³” or “<10⁻¹⁰” respectively.

The heavy metal ion visual image selection interface affordance 540 is operable to facilitate selection by a user of a particular visual image for preprocessing and analysis. In some implementations, the heavy metal ion visual image selection interface affordance 540 is a graphical user interface button element. In some implementations, the heavy metal ion visual image selection interface affordance 540 begins an image capture by the magnification device 320, or a camera associated with the electronic device 302.

The heavy metal ion visual image processing interface affordance 540 is operable to facilitate analysis by a user of a particular visual image for detecting one or more of presence and concentration of heavy metal ions. In some implementations, the heavy metal ion visual processing interface affordance 540 is a graphical user interface button element. In some implementations, the heavy metal ion visual image processing interface affordance 540 begins an image processing method in accordance with one or more of methods 700 and 800, and in accordance with the system 300 and the system memory 400.

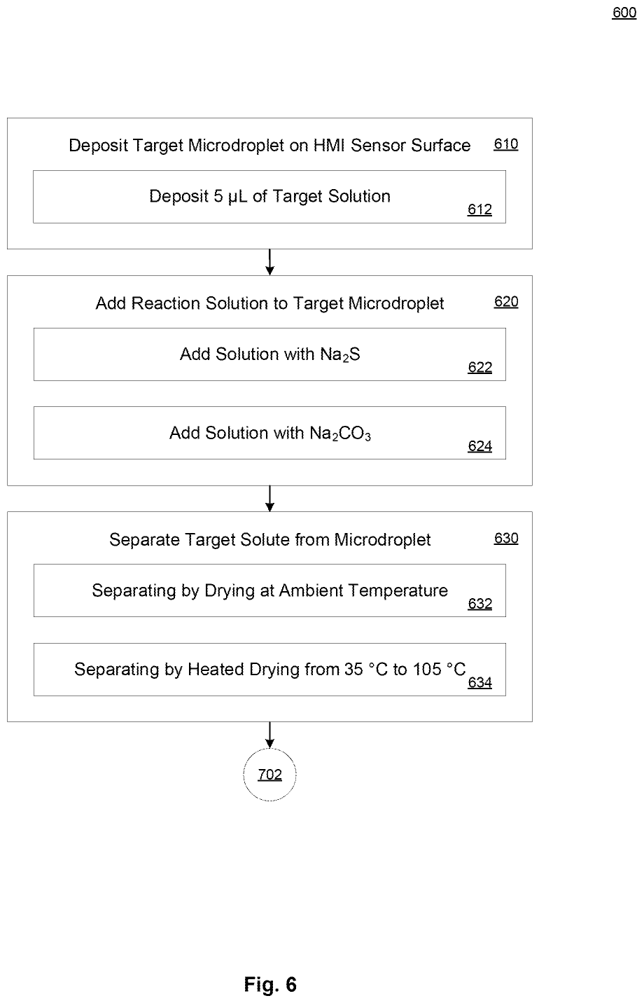

FIG. 6 illustrates an example method of depositing a target analyte at a portable sensor device for rapid detection of heavy metal ions, in accordance with present implementations. In some implementations, at least one of the example device 100 and the example system 300 performs method 600 according to present implementations. In some implementations, the method 600 begins at step 610.

At step 610, the example system deposits at least one target microdroplet on a heavy metal ion sensor surface. In some implementations, step 610 includes step 612. At step 612, the example system deposits approximately 5 microliters of target solution on the heavy metal ion sensor surface. The method 600 then continues to step 620.

At step 620, the example system adds a reaction solution to the target microdroplet. In some implementations, step 620 includes at least one of steps 622 and 624. At step 622, the example system adds a reaction solution including sodium sulfide to the target microdroplet. At step 624, the example system adds a reaction solution including sodium carbonate to the target microdroplet. In some implementations, steps 622 and 624 are combined into a single step, and the sodium sulfide and sodium carbonate are added to the microdroplet concurrently, simultaneously, or the like. The method 600 then continues to step 630.

At step 630, the example system separates a target solute from the target microdroplet. In some implementations, step 630 includes at least one of steps 632 and 634. At step 632, the example system separates the target solute from the target microdroplet by drying the target microdroplet at ambient temperature. At step 634, the example system separates the target solute from the target microdroplet by heating the target microdroplet to a temperature between 35° C. and 105° C. As one example, the target microdroplet can be heated to approximately 75° C. The method 600 then continues to step 702.

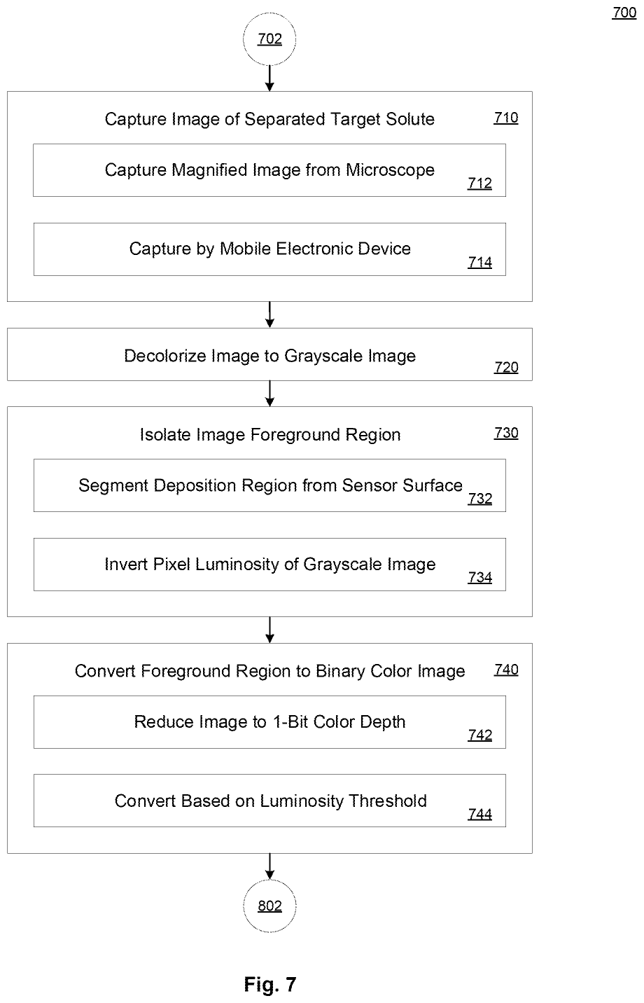

FIG. 7 illustrates an example method of preprocessing an image of a target analyte at a portable sensor device for rapid detection of heavy metal ions, further to the example method of FIG. 6. In some implementations, at least one of the example device 100 and the example system 300 performs method 700 according to present implementations. In some implementations, the method 700 begins at step 702. The method 700 then continues to step 710.

At step 710, the example system captures an image of the target solute separated from the microdroplet. In some implementations, step 710 includes at least one of steps 712 and 714. At step 712, the example system captures a magnified image from a microscope. At step 714, the example system captures the image by a mobile electronic device. In some implementations, the microscope is operatively coupled to a lens or a communication channel of the mobile electronic device. The method 700 then continues to step 720.

At step 720, the example system decolorizes the captured image to a grayscale image. The method 700 then continues to step 730.

At step 730, the example system isolates an image foreground region from the grayscale image. In some implementations, step 730 includes at least one of steps 732 and 734. At step 732, the example system segments a deposition region from a surrounding sensor surface region. At step 734, the example system inverts pixel luminosity of the grayscale image. The method 700 then continues to step 740.

At step 740, the example system converts the image foreground region to a binary color image. In some implementations, step 740 includes at least one of steps 742 and 744. At step 742, the example system. At step 744, the example system converts the foreground region to a binary color image based on a luminosity threshold. In some implementations, a luminosity threshold indicates a luminosity value representing a threshold between a “black” luminosity and a “white” luminosity. It is to be understood that any two colors, patterns, visual representations, or the like, can be associated with luminosity values variously satisfying and not satisfying the luminosity threshold. In some implementations, the luminosity threshold is predetermined to advantageously improve visual perceptibility of heavy metal ions against at least one of a sensor substrate and deposited reaction solute. The method 700 then continues to step 802.

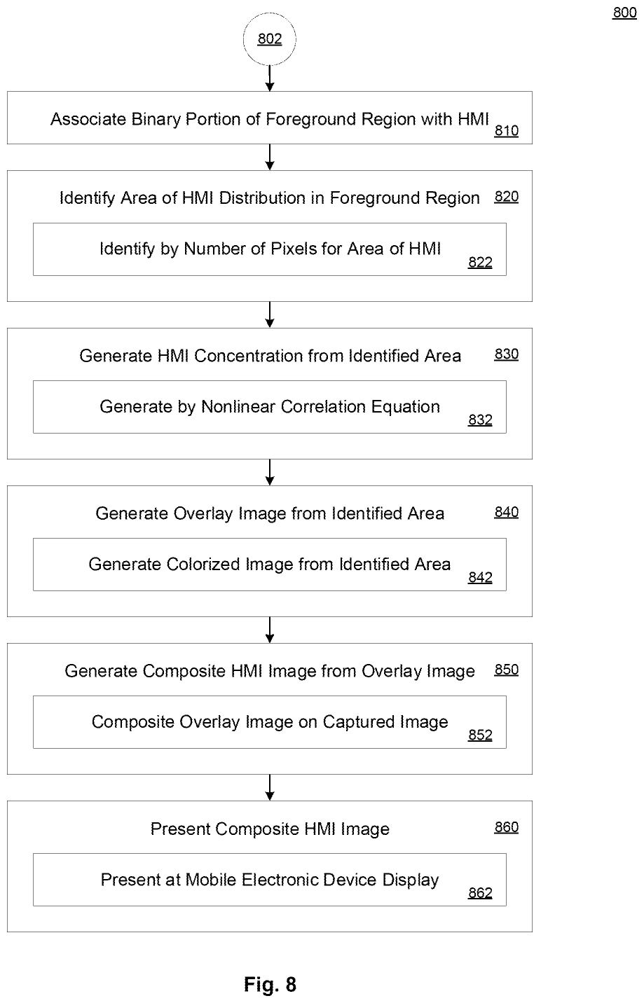

FIG. 8 illustrates an example method of sensing and presenting a concentration of a target analyte at a portable sensor device for rapid detection of heavy metal ions further to the example method of FIG. 7. In some implementations, at least one of the example device 100 and the example system 300 performs method 800 according to present implementations. In some implementations, the method 800 begins at step 802. The method 800 then continues to step 810.

At step 810, the example system associates a binary portion of the foreground region with the presence of heavy metal ions. In some implementations, the example system associates “black” or “white” pixels with the presence of heavy metal ions. The method 800 then continues to step 820.

At step 820, the example system identifies an area of heavy meal ion distribution in the foreground region. In some implementations, a distribution area is associated with an area ranging from 0.55 mm² to 0.90 mm² for a concentration of 10⁻³ M. In some implementations, step 820 includes step 822. At step 822, the example system identifies an area of heavy metal ion distribution in the foreground region by identifying a number of pixels associated with an area of distribution of the heavy metal ions. The method 800 then continues to step 830.

At step 830, the example system generates at least one heavy metal ion concentration based on the identified area. In some implementations, step 830 includes step 832. At step 832, the example system generates a heavy metal concentration based on the identified area of heavy metal ion distribution. In some implementations, step 830 includes step 832. At step 832, the example system generates a heavy metal concentration based on a nonlinear correlation between the identified area of heavy metal ion distribution and a predetermined concentration of heavy metal ions associated therewith. In some implementations, the nonlinear correlation includes a nonlinear correlation equation between identified area of heavy metal ion distribution and predetermined concentration of heavy metal ions. The method 800 then continues to step 840.

At step 840, the example system generates at least one overlay image based on the identified area of heavy metal ion distribution. In some implementations, step 840 includes step 842. At step 842, the example system generates a colorize image based on the identified area. The method 800 then continues to step 850.

At step 850, the example system generates a composite heavy metal ion image based on the overlay image. In some implementations, step 850 includes step 852. At step 852, the example system composites the overlay image over the captured image. The method 800 then continues to step 860.

At step 860, the example system presents the composite heavy metal ion image. In some implementations, step 860 includes step 862. At step 862, the example system presents the composite heavy metal ion image at a mobile electronic device display. In some implementations, the method 800 ends at step 860.

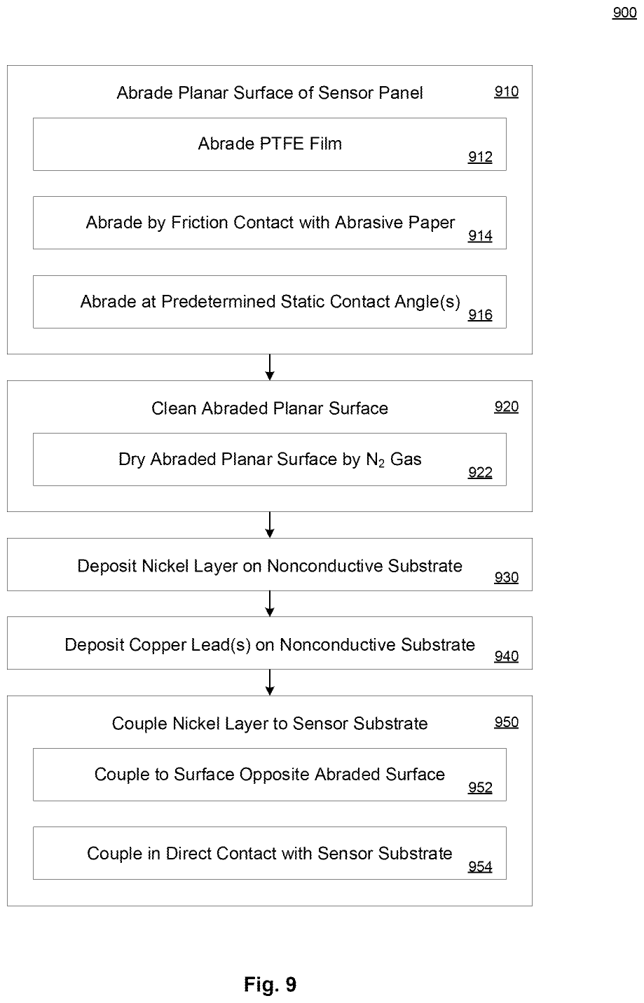

FIG. 9 illustrates an example method of manufacturing a portable sensor device for rapid detection of heavy metal ions, in accordance with present implementations. In some implementations, the example device 100 is manufactured by method 900 according to present implementations. In some implementations, the method 900 begins at step 910.

At step 910, the example system abrades a planar surface of a sensor panel. In some implementations, step 910 includes at least one of steps 912, 914 and 916. At step 912, the example system abrades a PTFE film. At step 914, the example system abrades the planar surface of the sensor panel by friction contact with abrasive material. As one example, the PTFE can have a contact angle hysteresis of approximately or substantially 51 degrees. This de-wetting property can enable sample droplets to adhere on the substrate for easy manipulation while allowing substrate-water contact area to recede during droplet evaporation for additional analyte enrichment. Thus, in some implementations, increasing surface particle density consequently promotes sensitive visual read-out of dark particle clusters. In some implementations, the abrasive material is or includes sandpaper, a sanding block, an industrial grinder, or the like. At step 916, the example system abrades the planar surface of the sensor panel at a predetermined contact angle. In some implementations, the predetermined contact angle is approximately 150 degrees from the planar surface of the sensor panel. As one example, the contact angle can be 150 degrees or greater. The method 900 then continues to step 920.

At step 920, the example system cleans the abraded planar surface. In some implementations, step 920 includes step 922. At step 922, the example system cleans the abraded planar surface by contact with nonreactive gas, noble gas, or the like. As one example, the gas can be or include nitrogen gas. The method 900 then continues to step 930.

At step 930, the example system deposits a metallic layer on a nonconductive substrate. As one example, the metallic layer can include nickel. As another example, the nonconductive substrate can include glass. The method 900 then continues to step 940.

At step 940, the example system deposits one or more metallic leads on the nonconductive substrate. As one example, the metallic leads can include copper. The method 900 then continues to step 950.

At step 950, the example system couples the metallic layer to the sensor substrate. In some implementations, step 950 includes at least one of steps 952 and 954. At step 952, the example system couples the metallic layer to a planar surface of the sensor panel opposite to the abraded surface. At step 954, the example system couples the metallic layer in direct contact with the sensor substrate. In some implementations, the method 900 ends at step 950.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A sensor device comprising:
a superhydrophobic sensor panel having an abraded first planar surface and a second planar surface opposite to the first planar surface; and
a metallic heating element adjacent to the second planar surface of the superhydrophobic sensor panel.

2. The device of claim 1, further comprising:
a nonconductive substrate adjacent to the metallic layer, wherein the metallic layer is adjacent to the superhydrophobic sensor layer at a first planar surface thereof, and adjacent to the nonconductive substrate at a second planar surface thereof.

3. The device of claim 1, wherein the abraded first planar surface includes surface features each having contact angles of 150 degrees or greater.

4. The device of claim 1, wherein the superhydrophobic sensor panel comprises poly(tetrafluoroethylene) (PTFE).

5. The device of claim 1, wherein the metallic heating element comprises nickel.

6. The device of claim 1, further comprising:
a first electrical lead in contact with the metallic heating element at a first end of the metallic heating element; and
a second electrical lead in contact with the metallic heating element at a second end of the metallic heating element distal to the first end of the metallic heating element.

7. The device of claim 6, further comprising:
a battery device operatively coupled to the first electrical lead and the second electrical lead, and configured to cause heating of the metallic heating element by applying an electrical current thereto.

8. A method of detecting a concentration of heavy metal ions in a solution, the method comprising:
separating a target solute of a target microdroplet from the target micropdroplet;
identifying a distribution area of at least one heavy metal ion in an image of the target solute;
generating a heavy metal ion concentration quantity based on the distribution area;
generating a composite image including an indication of the distribution area; and
presenting an indication of at least one of the heavy metal ion concentration quantity and the composite heavy metal ion image.

9. The method of claim 8, further comprising:
depositing the target micropdroplet on an abraded first planar surface of a superhydrophobic sensor panel.

10. The method of claim 8, wherein the separating the target solute from the target micropdroplet comprises separating the target solute of the target microdroplet from the target micropdroplet by heating the target microdroplet to a temperature between 35 degrees C. and 105 degrees C.

11. The method of claim 8, wherein the at least one heavy metal ion is at least one of lead, nickel, chromium, cobalt, and copper.

12. The method of claim 8, further comprising:
adding a reaction solution including at least one of sodium sulfide and sodium carbonate to the target microdroplet.

13. The method of claim 8, further comprising:
capturing the image of the target solute at microscopic magnification by a microscopic imaging device.

14. The method of claim 8, wherein the identifying the distribution area of the heavy metal ion in the image of the target solute comprises identifying the distribution area of the heavy metal ion in the image of the target solute by identifying a number of pixels associates with the distribution area of the heavy metal ion in the image.

15. The method of claim 8, wherein the generating the heavy metal ion concentration quantity based on the distribution area comprises generating the heavy metal ion concentration quantity based on the distribution area by a nonlinear correlation between distribution area and heavy metal ion concentration.

16. A method of manufacturing a heavy metal ion sensor device, comprising:
abrading a first planar surface of a superhydrophobic sensor panel;
depositing a metallic layer on a nonconductive substrate; and
contacting the metallic layer to a second planar surface of the sensor panel opposite to the first planar surface.

17. The method of claim 16, wherein the abrading the first planar surface of the superhydrophobic sensor panel comprises abrading the first planar surface of the superhydrophobic sensor panel at contact angles of 150 degrees or greater.

18. The method of claim 16, wherein the abrading the first planar surface of the superhydrophobic sensor panel comprises abrading the first planar surface of the superhydrophobic sensor panel by friction contact at the first planar surface with an abrasive solid.

19. The method of claim 16, wherein the superhydrophobic sensor panel comprises poly(tetrafluoroethylene) (PTFE).

20. The method of claim 16, wherein the metallic layer comprises nickel.
